(19) United States
(12) Patent Application Publication
(10) Pub. No.: US 2017/0219554 A1
(43) Pub. Date: Aug. 3, 2017

(54) APPARATUS AND METHOD FOR DETECTING ANALYTES IN SOLUTION

(71) Applicant: Mara Nanotech New York, Inc., Bayside Hill, NY (US)

(72) Inventor: Heayeon Lee, New York, NY (US)

(21) Appl. No.: 15/419,797

(22) Filed: Jan. 30, 2017

Related U.S. Application Data
(60) Provisional application No. 62/288,439, filed on Jan. 29, 2016.

(51) Int. Cl. G01N 33/483; C12Q 1/00; G01N 27/327; C12Q 1/68

(52) U.S. Cl. CPC G01N 33/4836; C12Q 1/6834; C12Q 1/001; G01N 27/3276

(57) ABSTRACT

Electrochemical biosensor devices and methods of using such devices are provided for detecting low concentration of an analyte in a biological fluid sample. One exemplary embodiment of an electrochemical biosensor device includes a plurality of electrodes made of a buffer layer laid on a substrate layer, an electrode layer laid on the buffer layer, and a perforated insulator layer laid on the electrode layer, such that a plurality of nanowells are formed on the electrode layer and the dimensions of the nanowells are defined by the sizes of the perforations, walls of the nanowells are defined by the insulator layer, and the bottom floors of the nanowells are defined by an upper surface of the electrode layer. In some instances, the nanowells of the biosensors have a pitch ratio of 1:1. In other instances, the biosensors can detect analytes that are present in fM concentration range.

### INCORPORATION BY REFERENCE

This application further expressly incorporates by reference and makes a part hereof the U.S. Provisional Patent Application Ser. Nos. 62/288,439, filed Jan. 29, 2016.

### FIELD

The present disclosure is generally concerned with highly sensitive and selective biosensors and method of use of such biosensors. More particularly, the present disclosure concerns such biosensors that include a perforated insulation layer laid on an electrode of an electrochemical biosensor to form nanowells.

### BACKGROUND

Biosensors are used to detect the presence of biological molecules such as proteins, amino acids (e.g., DNA and/or RNA containing specific base sequences), or other organic molecules. Some of the examples of biosensors include pregnancy tests and glucose monitoring sensors. These biosensors can detect biomolecules such as human chronic gonadotropin (hCG) or glucose that are present in bodily fluids such as blood or urine.

In order to detect specific analytes (e.g., biological molecules), biosensors may contain an analyte-binding surface where probes specific for an analyte (e.g., single-strand DNA or antibody specific for the target molecule) are immobilized to the analyte-binding surface. Different types of biosensors using distinct scientific principles have been developed that can detect presence of specific biological molecules.

Examples of different types of biosensors include electrochemical biosensors, nano-cantilever biosensors, and micro- or nano-electromechanical systems (MEMS/NEMS). Like other types of biosensors, electrochemical biosensors comprise an analyte-binding surface that is capable of interacting with and/or binding to specific biomolecules (e.g., a specific protein or a specific sequence of DNA). In particular, electrochemical biosensors use the principle of electrochemical analysis to detect specific analytes, where chemical response to an electrical excitation applied to a system is measured and analyzed to detect whether an analyte is bound to the surface of an electrode. Unlike nano-cantilever biosensors and MEMS/NEMS, electrochemical biosensors' signals can be directly detected by an electronic device for analysis, allowing for fast diagnosis.

Potential future applications for electrochemical biosensors include diagnosis in traditional medical and healthcare setting (e.g., blood and/or urine sample testing for specific biological molecules); medical diagnosis non-hospital setting (e.g., military use in combat zone and/or self-administered consumer diagnostics), non-medical detection of biological and/or small molecule detection (e.g., water quality testing, environmental testing, quality control and/or quality assurance testing in food industry); companion diagnostics for pharmaceutical therapeutics; research applications where detection of small molecules are required; and/or other settings or circumstances where detection of biological molecules is needed. A person skilled in the art will appreciate that, although the present disclosure is called “biosensors,” its application is not limited to detection of biological molecules. In other words, the present disclosure may be used for detection of other small non-biological (e.g., inorganic, metallic, solute, electrolyte, and/or elemental) molecules. In addition, although examples provided here consist of detection in fluidic and/or aqueous milieu, one skilled in the art will appreciate that the present disclosure may be used to detect small molecules in other fluidic milieu such as in oil, solvents, gas, and/or colloidal solutions.

In order for electrochemical biosensors to be adapted widely for a broad range of applications, the biosensors must be highly sensitive and selective, and cost of manufacturing of such sensor must be competitive. Electrochemical biosensors with significantly improved sensitivity and selectivity may enable miniaturization of such devices, which in turn may reduce the production cost and further contribute to adoption of electrochemical biosensors for a wide range of applications.

To the best of the applicant's knowledge, currently, there are no electrochemical sensors that can detect multiple analytes that are present in fM-range in biological samples with high selectivity. Accordingly, there is a need for electrochemical biosensors that can detect multiple analytes that are present in fM range in biological samples. There is also a need for such biosensors that can be reliably and stably produced in large scale at a low cost.

### SUMMARY

Electrochemical biosensor devices and methods of using such devices are provided for detecting low concentration of an analyte in a biological fluid sample. One exemplary embodiment of an electrochemical biosensor device includes a plurality of electrodes made of a buffer layer laid on a substrate layer, an electrode layer laid on the buffer layer, and a perforated insulator layer laid on the electrode layer, such that a plurality of nanowells are formed on the electrode layer and the dimensions of the nanowells are defined by the sizes of the perforations, walls of the nanowells are defined by the insulator layer, and the bottom floors of the nanowells are defined by an upper surface of the electrode layer. In some instances, the nanowells of the biosensors have a pitch ratio of 1:1. In other instances, the biosensors can detect analytes that are present in fM concentration range.

In some embodiments, the electrochemical biosensor can include glass substrate layer, silicon substrate layer, silicon dioxide insulator layer, titanium buffer layer, chromium buffer layer, and/or gold electrode layer.

In yet other embodiments, the electrochemical biosensor can have perforated insulator layer, wherein the perforations (e.g., bores and/or holes) may define dimensions of nanowells such that the nanowells are cylindrical in shape. In yet some other embodiments, the nanowells have circular openings with a diameter of about 230 nm, 100 nm, and/or 50 nm. In further yet other embodiments, the nanowells have pitch ratio of about 1:5, about 1:3 and/or about 1:1.

In some embodiments, the electrochemical biosensor can operate in conjunction with an electronic device, whereby the electrochemical biosensor is capable of sending signals to the electronic device such that one or more electrochemical reaction parameters between the electrode containing a reference sample and the electrode containing a test sample can be detected by the electronic device using the signals to determine whether an analyte is present in the test sample. In an exemplary embodiment, the electrochemical reaction comprises oxidation reaction and reduction reaction. In a further exemplary embodiment, the parameters comprises variation in redox current.

In some other embodiments, the electrochemical biosensor can be used to detect analytes in sample solutions by (1) applying the test sample to sensing electrodes of the electrochemical biosensor to allow binding of any analytes that may be present in the test sample; (2) rinsing the sensing electrodes with an appropriate buffer to wash away any unbound and/or non-specifically bound analytes and/or non-analytes from the sensing electrodes; (3) applying electric current to the sensing electrode in such a way to cause chemical changes to the sensing electrode; (4) measuring electrochemical properties of the sensing electrode using an electronic device; and (5) analyzing difference in electrochemical properties between the test sample and the reference sample to determine presence of an analyte on the sensing electrode. In an exemplary embodiment, the electrochemical properties of the sensing electrode is measured using cyclic voltammetry.

### BRIEF DESCRIPTION OF THE DRAWINGS

FIGS. 1A-1D: Photographic and microscopy images of an embodiment of present disclosure;

FIG. 2: A cross-sectional schematic diagram of a sensing electrode;

FIG. 3: A perspective schematic diagram of an embodiment of present disclosure, illustrating individual sensing electrodes being configured to detect different analytes;

FIG. 4 A schematic diagram of how a biosensor may be used in conjunction with a potentiostat and an electronic device to detect analytes in a sample;

FIGS. 5A-5C, 6A-6C: A schematic diagram and representative data illustrating exemplary pitch ratios of nanowells and the effect of varying pitch ratios on biosensor sensitivity;

FIG. 7: Representative data illustrating detection of varying concentration of DNA analytes in a solution using an embodiment of present disclosure.

### DETAILED DESCRIPTION

Certain exemplary embodiments will now be described to provide an overall understanding of the principles of the structure, function, and use of the devices disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in collection with one exemplary embodiment may be combined with the features of other embodiments. Such modifications and variations are intended to be included within the scope of the present application.

Additionally, the figures are not necessarily to scale and, to the extent that linear or circular dimensions are used in the description of the disclosed devices and methods, such dimensions are not intended to limit the types of shapes and sizes that can be used in conjunction with such devices and methods. A person skilled in the art will recognize that an equivalent to such linear and circular dimensions can easily be determined for any geometric shape. Still further, sizes and shapes of the devices, and the components thereof, can depend at least on the anatomy of the subject in which the device will be used, the size and shape of components with which the device will be used, and the methods and procedures in which the device will be used.

Furthermore, while the exemplary embodiments provided herein describe use of the device in detecting biomolecules (e.g., proteins and/or nucleic acid molecules), a person skilled in the art will recognize that the device may be adopted to be used to detect presence of non-biological molecules and/or samples that are not biological samples. As an example, presence of inorganic material may be detected using the present invention for water quality testing, environmental testing and/or quality control/quality assurance testing in other industrial settings).

In order for an electrochemical sensor to be adopted in a wide range of applications such as diagnosis in traditional medical, pharmaceutical, and/or healthcare settings (e.g., blood and/or urine sample testing for specific biological molecules), medical diagnosis in non-hospital setting (e.g., military use in combat zone, self-administered consumer diagnostics such as pregnancy test or blood glucose monitoring), non-medical detection of biological and/or small molecule detection (e.g., water quality testing, environmental testing, quality control and/or quality assurance testing in food industry), companion diagnostics for pharmaceutical therapeutics; research applications where detection of small molecules are required, and/or other settings or circumstances where detection of biological molecules is needed, the electrochemical sensor must be sensitive (i.e., being able to detect low concentrations of analyte), selective (i.e., being able to distinguish and differentiate target analytes in the presence of other components), easy to use (i.e., simple to operate, requires small amounts of test samples), and readily available to users (i.e., able to manufacture scalably, in large quantities, and/or at a low cost).

The present disclosure is directed to highly sensitive and highly selective electrochemical biosensors made using components that are more resilient and stable compared to past electrochemical biosensors.

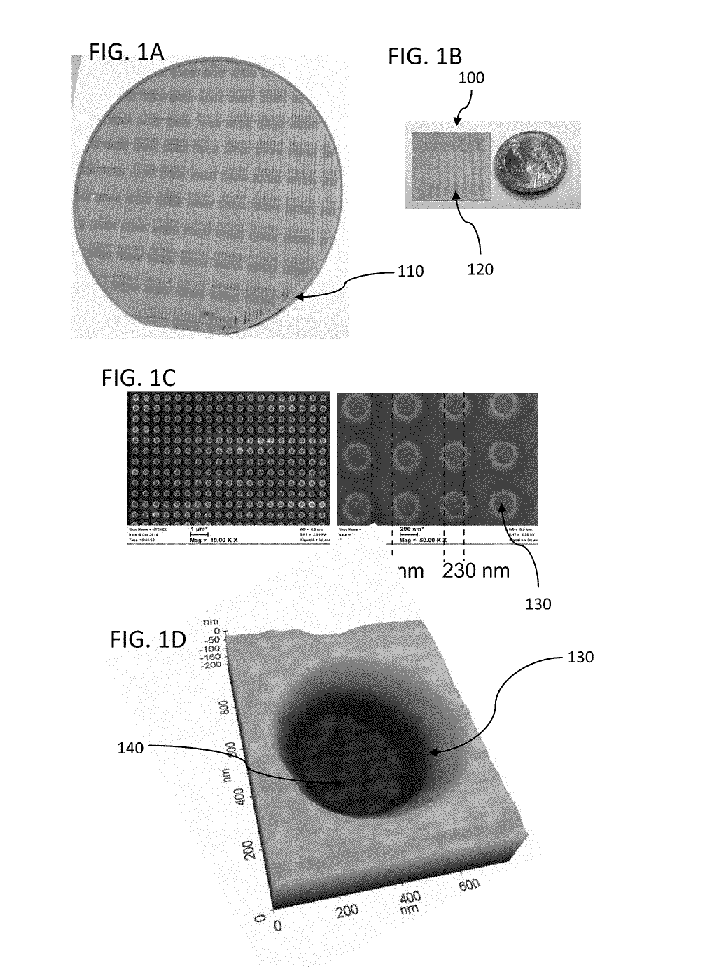

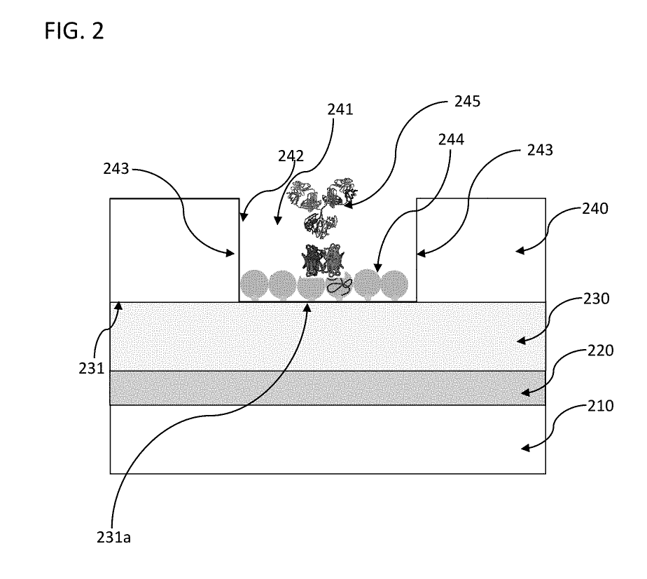

FIG. 1A-1D illustrate photographic and microscopy images of one embodiment of an electrochemical biosensor 100. As illustrated in FIG. 1A, a plurality of one embodiment of present disclosure 100 may be produced on a single substrate layer 110, such as a glass or silicon substrate layer (e.g., a wafer). FIG. 1B is a photographic image of a single electrochemical biosensor 100 comprising multiple sensing electrodes 120. FIG. 1C is a scanning electron microscopy image of the sensing electrode of FIG. 1B, comprising a plurality of nanowells 130 having a pitch ratio (ratio between the diameter of the nanowell openings and the shortest distance between neighboring nanowells) of approximately 1:1, and the nanowell opening diameter of approximately 230 nm. In other embodiments, the nanowell pitch ratio and/or the nanowell opening diameter may be of different value, as disclosed in later portions of the present disclosure. FIG. 2D is a perspective atomic force microscopy image of a single nanowell 130, showing cylindrical dimensions of a nanowell 130 having a bottom floor 140 that is defined by the top surface of an electrode layer.

FIG. 2 illustrates cross-sectional schematic representation of a portion of a sensing electrode of FIG. 1. In one embodiment, the sensing electrode may comprise a glass or silicon substrate layer 210, a buffer layer 220, laid on the substrate layer, an electrode layer 230 laid on the substrate layer, and an insulator layer 240 laid on the electrode layer.

In one embodiment, the substrate layer 210 may be made of glass. The substrate layer may also comprise silicon, silicon dioxide (e.g., quartz), borosilicate, and/or other glass compositions used in semiconductor manufacturing. In other embodiments, the glass or silicon substrate layer 210 may be a circular wafer. In yet other embodiments, the glass or silicon substrate layer 210 may be configured to accommodate a plurality of electrochemical biosensors, as illustrated in FIG. 1A.

The buffer layer 220 of the sensing electrode 120 may provide enhanced bonding of the electrode layer 230 to the substrate layer 210 thereby minimizing risk of the electrode layer 230 detaching from the substrate layer 210. In other words, the buffer layer 220 allows the electrode layer 230 and the substrate layer 210 to form a tighter seal. Such enhanced bonding or formation of seal between the electrode layer 230 and substrate layer 210 may enable easier and more reliable manufacturing of the electrochemical biosensor and/or reduce cost of manufacture. In one preferred embodiment, the buffer layer may comprise titanium, chromium, and/or alloys of titanium or chromium.

The insulator layer 240 of the sensing electrode 120 may be perforated, such that the insulator layer 240 comprises a plurality of bores 241 (i.e., holes). The plurality of bores is also illustrated in FIG. 1C, the bores forming the nanowells 130. In a preferred embodiment, the bores 241 define the internal dimensions of a plurality of nanowells 242. In some embodiments, the bores 241 are cylindrical in shape, as shown in FIG. 1D, such that the nanowells 242 whose dimensions are defined by the bores 241 have a circular opening and the insulator layer form the walls 243 of the cylindrical nanowells 242. Because the perforated insulator layer 240 is laid on the electrode layer 230, by virtue of the relative positions between the two layers, the electrode layer's top surface 231 that is not covered by the insulator layer 240 may form the bottom surface of the nanowell 231a. In one preferred embodiment, the insulator layer 240 of the sensing electrode may comprise silicon nitride (Si₃N₄), because silicon nitride is more resilient and stable compared to certain alternatives, such as organic or inorganic polymers. In another preferred embodiment, the insulator layer 240 of the sensing electrode may comprise silicon dioxide (SiO₂). Features such as resilience and stability of silicon nitride may enable a more reliable and consistent manufacturing of the sensing electrode portion of the present disclosure, resulting in reduced occurrences of defective products and reduced cost of manufacture.

In some embodiments, where the opening of the nanowell is circular, the diameter of the circular opening of the nanowells 242 may be less than 1000 nm. In other embodiments, the diameter of the circular opening of the nanowells 242 may be less than 300 nm. In yet other embodiments, the diameter of the circular opening of the nanowells 242 may be approximately 230 nm, 100 nm, and/or 50 nm. Although the embodiments described above has nanowells 242 that are cylindrical in shape with a circular opening, a person skilled in the art will recognize that the nanowells 242 may have various other opening shapes, such as rectangular, oval, and/or polygonal shapes. In these embodiments having nanowells 242 with various other opening shapes, the dimension of the opening may be less than 1000 nm or 300 nm, or may be approximately 230 nm, 100 nm, and/or 50 nm. In addition, a person skilled in the art will also recognize that the present invention is not limited to the compositions and structure described above, but may also include compositions and structure with similar characteristics, or improved characteristics.

In other embodiments, the bottom surfaces 231a of the nanowells 242 (e.g., top surface of the electrode layer that is not covered by the insulator layer) may comprise probe molecules 245 that are capable of binding with specific analytes. As an example, as shown in FIG. 2, biotinylated antibodies 245 specific for an analyte may be immobilized to the bottom surfaces 231a of the nanowells by using an intermediary binding molecule 244 such as avidin or streptavidin. A person skilled in the art will appreciate that other well-known methods of immobilizing analytes probes 245 can be incorporated into present disclosure, and are within the scope of present invention, as discussed below. In yet another preferred embodiment, the insulator layer 240 with a plurality of bores 241 may restrict binding of an analyte to its probes 245 to the bottom surfaces 231a of the nanowells 242, while preventing binding and/or aggregation of the analyte to the insulator layer 240.

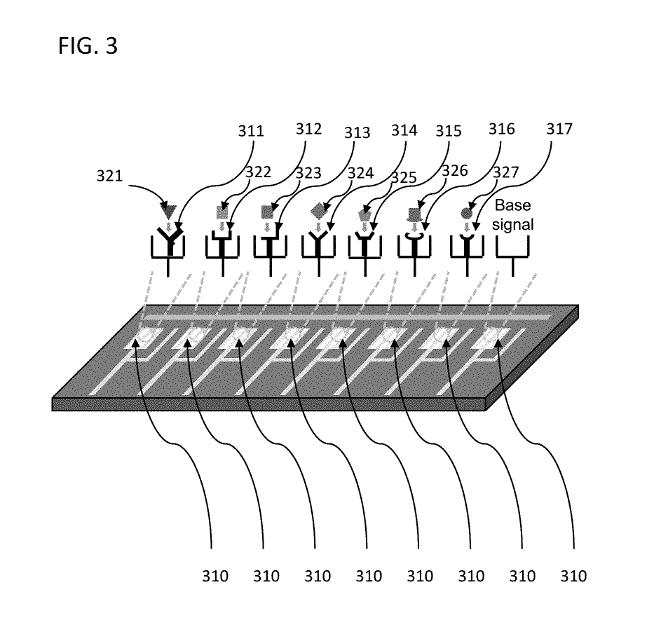

FIG. 3 illustrates a perspective schematic representation of one embodiment of an electrochemical biosensor 100, wherein each individual sensing electrode 310 is coated with specific analyte probes 311-317 (e.g., antibody) such that different analytes 321-327 (e.g., proteins) can bind to the different probes 311-317 (e.g., by protein-protein interaction, DNA-DNA hybridization and/or other intermolecular binding) that are immobilized on the individual sensing electrode 310. The term “immobilized” means binding a specific analyte probe (e.g., 311) to the surface of the sensing electrode 310, for example, by binding the probe to the electrode surface by covalent bonding, hydrogen bonding, ionic bonding, and/or Van der Walls forces. In one preferred embodiment, the electrochemical biosensor comprises a plurality of electrodes 310 capable of sensing very low amounts of analytes (e.g., less than 1000 fM in concentration, less than 500 fM in concentration, less than 100 fM in concentration, less than 10 fM in concentration and/or less than 1 fM in concentration).

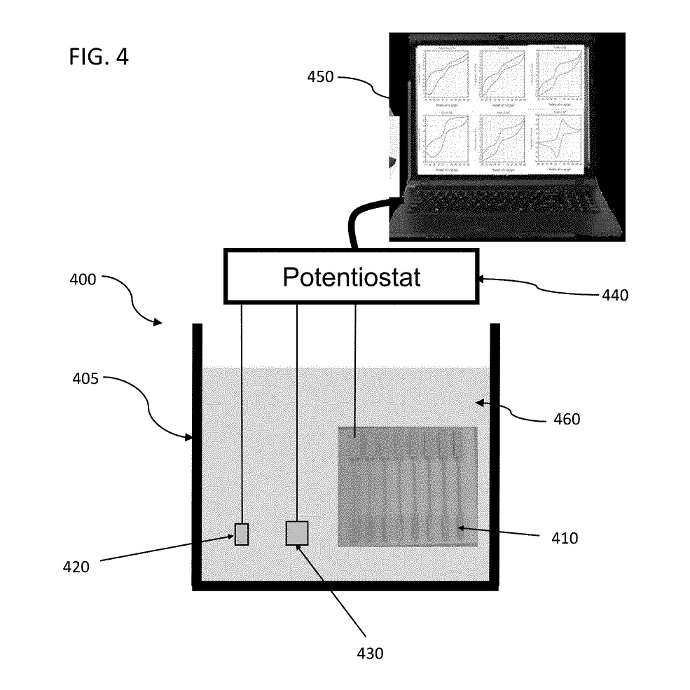

FIG. 4 illustrates a schematic representation of how one preferred embodiment of an electrochemical biosensor 410 may be used in conjunction with a potentiostat 440 and an electronic device 450 to detect analytes in a sample. In one embodiment, a user (e.g., a consumer, a laboratory personnel, a nurse, a doctor, a computer system, a machine or robotic device that uses the present disclosure as a component or step) may use the present disclosure to measure analytes in samples by performing the following steps: (1) applying test samples to the sensing electrodes to allow binding of analytes to analyte probes; (2) rinsing the sensing electrodes to remove unbound and/or non-specifically bound molecules (analytes and/or non-analytes) from the sensing electrode, (3) performing electrochemical measurements and analysis on the sensing electrodes. A preferred embodiment of the present disclosure uses cyclic voltammetry to measure electrochemical properties of an analyte in solution, as shown in FIG. 4. In such an embodiment, an electrochemical biosensor (working electrode) 410 is used in conjunction with an electrochemical chamber 400, a potentiostat 440, and an electronic devices 450 such as a computing device (e.g., personal computer, server, laptop, smartphone, purpose-built electronic device, and/or any other device that may be capable of receiving and analyzing electrical signals from the present disclosure). The electrochemical chamber 400 comprising a reservoir 405, a reference electrode 420, a counter electrode 430, and working electrode (the working electrode being a component of the electrochemical biosensor) 410. The reference electrode 420, counter electrode 430 and working electrode 410 may be submerged in a solution of electrolyte 460 such that when an electrical excitation is applied to the system 400, the electrical excitation causes chemical responses (e.g., oxidation and/or reduction reactions) that can be detected and analyzed by an electronic device 450. More specifically, when a current is applied to flow between the working electrode 410 and counter electrode 430, electric potential of the working electrode 410 relative to the reference electrode 420 can be controlled by the potentiostat 440. In this instance, the electric potential between the working electrode 410 and the reference electrode 420 can be measured accurately, irrespective of electric current resulting from electrode reaction. A person skilled in the art will appreciate that other alternative electrochemical measurement methods may also be adopted to the present disclosure, and thus are within the scope of the present disclosure.

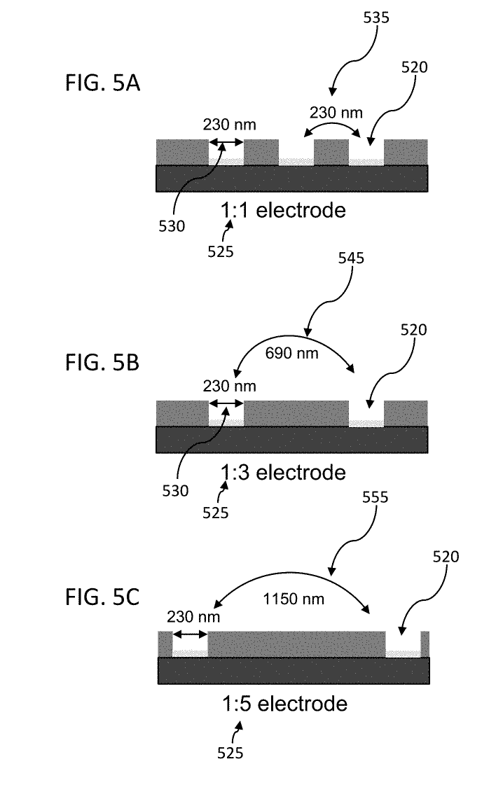

FIGS. 5A-5C illustrate cross sectional schematic representation of various embodiments of the present disclosure having different distribution of nanowells 520 on a sensing electrode 500. Distribution of the nanowells 520 is expressed in terms of pitch ratios 525 between neighboring nanowells 520. A pitch ratio 525 is defined by the ratio between the opening diameter 530 of a nanowell 520 and the nearest distance 535, 545, 555 between two neighboring nanowells. Another illustration of the nearest distance 535, 545, 555 can be found in FIG. 1B, where the nearest distance between neighboring nanowells is shown to be 230 nm.

FIG. 5A illustrates a preferred embodiment where the pitch ratio 525 of the nanowells 520 is 1:1. In this embodiment, the nanowell 520 opening has a diameter 530 of 230 nm and the shortest distances 535 between the neighboring nanowells 520 is 230 nm. Hence, the ratio between the nanowell opening diameter 530 and the shortest distances 535 between the neighboring nanowells is 230 nm:230 nm, or 1:1. FIG. 5B illustrates yet another embodiment where the pitch ratio 525 is 1:3. In this embodiment, the nanowell 520 opening has a diameter 530 of 230 nm and the shortest distance 545 between the neighboring nanowells 520 is 690 nm. Hence, the ratio between the nanowell opening diameter 530 and the shortest distance 545 between neighboring nanowells is 230 nm:690 nm, or 1:3. FIG. 5C illustrates still yet another embodiment where the pitch ratio 525 is 1:5. In this embodiment, the nanowell 520 opening has a diameter 530 of 230 nm and the shortest distance 550 between the neighboring nanowells 520 is 1150 nm. Hence, the ratio between the nanowell opening diameter 530 and the shortest distance 550 between neighboring nanowells is 230 nm:1150 nm, or 1:5. A person skilled in the art will recognize that, as discussed earlier in the present disclosure, that these are exemplary embodiments, and other sizes of nanowells and/or pitch ratios are also within the scope of the present disclosure. For example, the nanowell opening diameter can be approximately 1000 nm, approximately 500 nm, approximately 100 nm, approximately 50 nm, approximately 20 nm or less than 20 nm. Similarly, pitch ratios can range from any ratio between 100:1 to 1:100, including 50:1, 10:1, 5:1, 3:1, 1:1, 1:3, 1:5, 1:10 or 1:50. In addition, although the nanowell distribution in the embodiments are in a grid-like uniform patterns, other similar patterns or non-uniform distribution of nanowells and/or other similar, equivalent, and/or further improvements to the arrangement that can further improve sensitivity and/or specificity of the present invention are within the scope of the present disclosure.

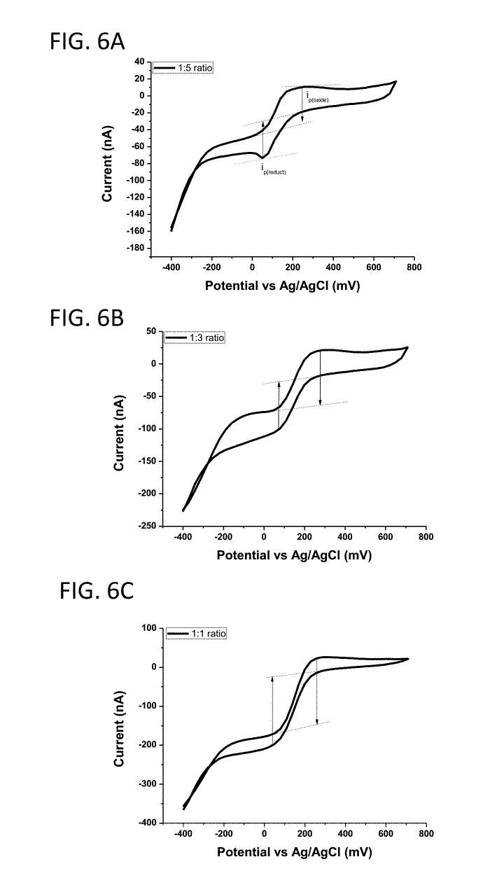

FIGS. 6A-6C illustrate differences in sensitivity of electrochemical biosensors having different nanowell pitch ratios (i.e., distances between neighboring nanowells). Generally, varying pitch ratios between nanowells results in changes in detection sensitivity. Specifically, cyclic voltammetry measurement of the present invention was performed wherein FIG. 6C shows highest sensitivity (cathodic peak of 1.75×10⁻⁷ nA) for biosensors having nanowell pitch ratio of 1:1, compared to FIG. 6A (cathodic peak of 4.4×10⁻⁸, biosensors having nanowell pitch ratio of 1:5) or FIG. 6B (cathotic peak of 7.9×10⁻⁸, biosensors having nanowell pitch ratio of 1:3). One skilled in the art will appreciate that both nanowell 420 opening sizes, pitch ratio, and/or other dimensional, topographical, and/or physical attributes of the nanowells may affect sensitivity of the present disclosure, and variable configurations of nanowells is within the scope of the present disclosure.

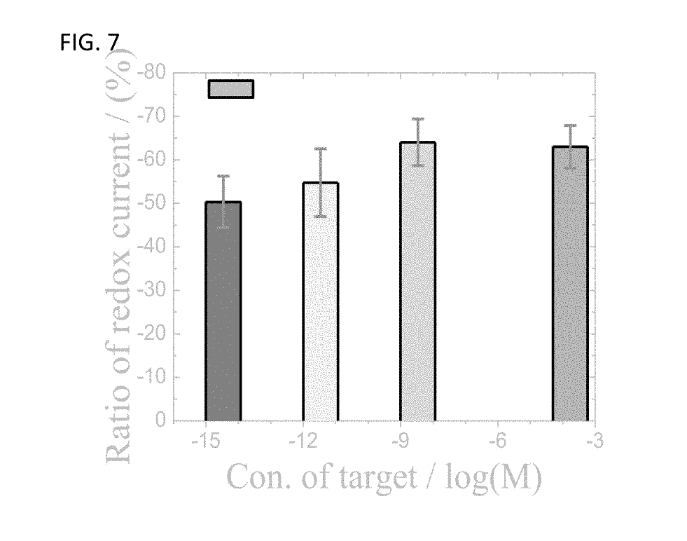

FIG. 7 shows representative data showing the ratio of redox current measured by an embodiment of the present disclosure to detect different concentrations of DNA analytes. Detection of the presence of analytes in samples having analyte concentrations ranging from mM (10⁻⁴ M) range to fM (10⁻¹⁵ M) range was performed by measuring changes in redox current, represented in percentages. This example illustrates that an embodiment of the present disclosure can detect fM range of DNA analytes by detecting statistically significant ratio of redox current.

While the foregoing description has been directed to specific embodiments, it will be apparent that other variations and modifications may be made to the described embodiments, with the attainment of some or all of their advantages. Accordingly this description is to be taken only by way of example and not to otherwise limit the scope of the embodiments herein. Therefore, it is the object of the appended claims to cover all such variations and modifications as come within the true spirit and scope of the embodiments described herein. Finally, all publications and references cited herein are expressly incorporated by reference in their entirety.

What is claimed is:

1. An electrochemical biosensor device for sensing presence of a molecule in solution, the device comprising:
a substrate layer; and
a plurality of electrodes, the electrodes further comprising:
a buffer layer laid on the substrate layer, the buffer layer configured to provide bonding of the plurality of electrodes to the substrate layer;
an electrode layer laid on the buffer layer, the electrode layer configured to provide binding sites for analytes; and
an insulator layer having a plurality of bores, the insulator layer laid on the electrode layer and the insulator having a plurality of bores configured to form a plurality of nanowells having side walls that are defined by the insulator layer and having bottom floors that are defined by a top surface of the electrode layer that is not covered by the insulator layer,
wherein an analyte probe is immobilized to the bottom floors of the nanowells, the analyte probe configured to bind to an analyte, and
wherein the insulator layer is configured to substantially confine binding of the analyte to the top surface of the electrode layer that define the bottom floors of the plurality of nanowells.

2. The electrochemical biosensor device according to claim 1,
wherein the substrate layer comprises glass.

3. The electrochemical biosensor device according to claim 1,
wherein the substrate layer comprises silicon.

4. The electrochemical biosensor device according to claim 1,
wherein the insulator layer comprises silicon nitride.

5. The electrochemical biosensor device according to claim 1,
wherein the insulator layer comprises silicon dioxide.

6. The electrochemical biosensor device according to claim 1,
wherein the buffer layer comprises titanium.

7. The electrochemical biosensor device according to claim 1,
wherein the buffer layer comprises chromium.

8. The electrochemical biosensor device according to claim 1,
wherein the electrode layer comprises gold.

8. The electrochemical biosensor device according to claim 1,
wherein the nanowell is cylindrical in shape and has a circular nanowell opening with a diameter of about 230 nm.

9. The electrochemical biosensor device according to claim 1,
wherein the nanowell is cylindrical in shape and has a circular nanowell opening with a diameter of about 100 nm.

10. The electrochemical biosensor device according to claim 1,
wherein the nanowell is cylindrical in shape and has a nanowell opening with a diameter of about 50 nm.

11. The electrochemical biosensor device according to claim 1,
wherein the pitch ratio between the plurality of nanowells is less than 1:5.

12. The electrochemical biosensor device according to claim 1,
wherein the pitch ratio between the plurality of nanowells is less than 1:3.

13. The electrochemical biosensor device according to claim 1,
wherein the pitch ratio between the plurality of nanowells is about 1:1.

14. The electrochemical biosensor device according to claim 1,
wherein the device is capable of sending signals to an electronic device, such that differences in one or more electrochemical reaction parameters between the electrode containing a reference sample and the electrode containing a test sample can be detected by the electronic device using the signals to determine whether the analyte is present in the test sample.

15. The electrochemical biosensor device according to claim 9,
wherein the electrochemical reaction comprise oxidation reaction and reduction reaction.

16. The electrochemical biosensor device according to claim 9,
wherein the parameters comprise variation in redox current.

17. Method of detecting an analyte in a test sample using the biosensor device according to claim 1, the method comprising steps of:
applying the test sample to the sensing electrode of the electrochemical biosensor device in such a manner that an analyte that may be present in the test sample is able to bind to the analyte probe;
rinsing the sensing electrodes with an appropriate buffer in such a manner that washes away non-bound and/or non-specifically bound analytes and non-analytes to be removed from the sensing electrode;
applying electric current to the sensing electrode in such a way to cause chemical changes to the sensing electrode;
measuring electrochemical properties of the sensing electrode using an electronic device; and
analyzing differences in electrochemical properties between the test sample and the reference sample to determine presence of an analyte on the sensing electrode.

18. The method according to claim 12 wherein,
electrochemical properties of the sensing electrode is measured using cyclic voltammetry.
